(19) United States
(12) Patent Application Publication
(10) Pub. No.: US 2022/0395833 A1
(43) Pub. Date: Dec. 15, 2022

(54) MICROFLUIDIC CELL CULTURE SYSTEM

(71) Applicant: Mimetas B.V., Oegstgeest (NL)

(72) Inventors: Kristina BISHARD, Oegstgeest (NL); Julia GRASEGGER, Oegstgeest (NL); Sebastiaan Johannes TRIETSCH, Oegstgeest (NL)

(21) Appl. No.: 17/774,751

(22) PCT Filed: Nov. 9, 2020
(86) PCT No.: PCT/NL2020/050701

§ 371 Date: May 5, 2022

(30) Foreign Application Priority Data
Nov. 8, 2019 (NL) 2024202

(51) Int. Cl. B01L 3/00; C12M 3/06

(52) U.S. Cl. CPC B01L 3/502761; B01L 3/502776; C12M 23/16; B01L 2300/0829

(57) ABSTRACT

The present invention relates to a microfluidic cell culture system comprising at least one microfluidic structure, wherein the at least one microfluidic structure comprises a cell culture chamber, a first and second reservoir in fluid communication with each other via the cell culture chamber, wherein the microfluidic cell culture system further comprises a detachable seal for sealing the at least one microfluidic structure and wherein the microfluidic cell culture system is configured such that the first and second reservoir of the at least one microfluidic structure are in fluid communication with each other via a communication channel that does not comprise the cell culture chamber.

The present invention relates to a microfluidic cell culture system comprising at least one microfluidic structure. The present invention further relates to a method for transporting the microfluidic cell culture system of the present invention and a kit-of-parts for forming the microfluidic cell culture system of the present invention.

In vitro cell and tissue cultures are essential tools in research as model systems for studying normal, disease and drug- or toxin-induced physiology and biochemistry. Cell and tissue culture techniques involve the distribution of cells in an artificial environment, e.g. by using microfluidic cell culture systems, providing the necessary nutrients, ideal temperature, gases, pH and humidity to allow cells to grow and proliferate. In order to warrant consistency and reproducibility of obtained results it is crucial that conditions are stable. Typically, the microfluidic cell culture systems comprise a plurality of microfluidic structures. Each of the microfluidic structures comprises a cell culture chamber for culturing cells. In order to warrant the consistency and reproducibility the microfluidic structures comprised in the microfluidic cell culture system need to remain intact and consistent during the lifecycle of the product, i.e. the microfluidic cell culture system. It is however observed that for the microfluidic cell culture systems provided nowadays, a high percentage of the microfluidic structures comprised in the microfluidic cell culture systems are already unsuitable for first use once received by the end user, e.g. a laboratory technician. The percentage of microfluidic structures that are already unsuitable for first use may be significant, i.e. about 50%.

In order to reduce the number of microfluidic structure unsuitable for first use, the present invention provides hereto a microfluidic cell culture system comprising at least one microfluidic structure, wherein the at least one microfluidic structure comprises:
a cell culture chamber for holding a medium for culturing cells, the cell culture chamber comprising a microfluidic inlet opening and a microfluidic outlet opening;
a first reservoir in fluid communication with the cell culture chamber via the microfluidic inlet opening; and
a second reservoir in fluid communication with the cell culture chamber via the microfluidic outlet opening.

The microfluidic cell culture system of the present invention further comprises a detachable seal for sealing the at least one microfluidic structure. Such detachable seal may be any seal suitable for sealing the microfluidic structure. The detachable seal may be a flexible or non-flexible seal. Further, the detachable seal of the present invention may be a form-retaining seal, such as a lid. Alternatively, the detachable seal may be a formless seal, such as a foil or parafilm. It is further noted that the detachable seal may be a reusable seal (e.g. a reusable lid) or a disposable seal (e.g. a foil or parafilm). In an embodiment of the present invention the detachable seal is a non-flexible, form-retaining cover, such as a (transparent) plastic lid.

The detachable seal has the advantage that it allows for easy removal after the detachable seal is no longer necessary, e.g. after transport and upon arrival in the intended laboratory. The detachable seal allows for ease of use in sealing and unsealing the microfluidic structure, while providing protection when applied to the microfluidic structure.

In the microfluidic cell culture system of the present invention the first and second reservoir of the at least one microfluidic structure are in fluid communication with each other via the cell culture chamber. It was found that during transport the pressure fluctuations within the microfluidic structure and, in particular, the pressure differences in the reservoirs of the microfluidic structure can result in rupture, irregularities, disruptions or the like of extracellular matrix, cultured cells (e.g. aggregates) or cell culture medium, in the cell culture chamber of the microfluidic structure resulting in a microfluidic structure being unsuitable for further use. Examples of aspects that are influenced by the rupture, irregularities, disruptions or the like are barrier function characteristics of layers or tubules comprising cell cultures, or the viability of the cells comprised in the cell cultures.

In order to reduce the number of microfluidic structures being unsuitable for further use after transportation of the microfluidic cell culture system of the present invention, the microfluidic cell culture system is configured such that the first and second reservoir of the at least one microfluidic structure are in fluid communication with each other via a communication channel that does not comprise the cell culture chamber. It was found that by providing a microfluidic structure wherein the reservoirs are in fluid communication with each other via another communication channel than the cell culture chamber, the fluctuations in pressure during transport and any pressure difference build up between reservoirs of the microfluidic structure is neutralized by the communication channel. As a result, the content and configuration of the cell culture chamber of the microfluidic structure is not affected by any fluctuations in pressure or pressure difference build-up in the reservoirs connected to the cell culture chamber.

In order to absorb fluctuations in the pressure during transport of the microfluidic cell culture system of the present invention, the design of the cell culture chamber and the communication channel is such that the fluidic resistance through the communication channel between the first and second reservoir is lower than the fluidic resistance through the cell culture chamber between the first and second reservoir. The fluidic resistance is inversely related to the average cross-section of the communication channel and proportionally related to the length of the communication channel. Preferably the fluidic resistance through the communication channel is at least 5 times lower than the fluidic resistance through the cell culture chamber, more preferably at least 10 times lower, even more preferably at least 50 times lower. The design of the cell culture chamber and the communication channel may not be limited to structural design choices only. Although the resistance of the communication channel and the cell culture chamber may be favourably controlled by designing a communication channel having a larger diameter compared to the diameter of the cell culture chamber (e.g. the diameters of the microfluidic inlet and outlet openings), the resistance of both the communication channel and the cell culture chamber is further controlled by selecting a different medium for the communication channel having a lower viscosity compared to the medium selected for the cell culture chamber having a higher viscosity. It is further noted that the communication channel is of the multidirectional (e.g. bidirectional) type. In case an one-directional channel is used for the communication channel, the system is not able to adequately react on fluctuations in the pressure during transport. In a preferred embodiment of the present invention, the communication channel is configured to exchange a gaseous medium, such as nitrogen, oxygen or air, between the reservoirs of the microfluidic structure.

As used herein, the microfluidic cell culture system of the present invention may be any system comprised of at least one microfluidic structure, preferably comprised of a plurality of microfluidic structures. An example of such microfluidic cell culture system is a microfluidic chip, microfluidic microtiter plate, microtiter plate, microwell plate or multiwell plate. As used herein, the microfluidic structure may be a structure formed of a network of microfluidic channels. Such network of microfluidic channels may be a complex network of multiple microfluidic channels. However, the microfluidic structure of the present invention may also include relatively simple network of microfluidic channels, e.g. a network comprised of a few microfluidic channels or even a single microfluidic channel. The microfluidic structure of the present invention may also be referred to as a microfluidic cell culture unit. An example of such microfluidic structure is a single channel connecting two wells of a microwell plate.

Further, as used herein, the reservoirs of the microfluidic structure may be any kind of container-like structure being suitable for use in a microfluidic structure. Preferably the reservoirs of the present invention are able to receive or to discharge fluids injected into or extracted from the reservoirs, respectively, by an operator, e.g. laboratory technician. Further the reservoirs of the present invention are preferably designed such as to receive means for providing a pressure over the reservoir in order to push fluids injected into the reservoir through the cell culture chamber connected to the reservoir. Such mean for providing a pressure over the reservoir may include pressurizing means and may include a pipette. In an embodiment of the present invention, the reservoirs of the present invention are provided with an opening situated in the surface of the microfluidic structure facing the inner side of the detachable seal of the microfluidic cell culture system of the present invention.

The microfluidic structure of the present invention may further comprise one or more capillary pressure barriers, such as phaseguides, in order to provide one or more subvolumes inside the cell culture chamber and/or one or more microfluidic channels. Such capillary pressure barriers or phaseguides are known in the art, e.g. in WO 2010/086179 A2 and WO 2014/038943 A1. As will become apparent from the exemplary embodiments described hereinafter, the capillary pressure barrier is not to be understood as a wall or a cavity which can for example be filled with a droplet of cell culture medium comprising one or more cells or cell aggregates, but consists of or comprises a structure which ensures that such a droplet does not spread due to the surface tension. This concept is referred to as meniscus pinning. As such, stable confinement of such a droplet to a subvolume, created by the capillary pressure barrier in the microfluidic structure, can be achieved. In one example, the capillary pressure barrier may be referred to as a confining phaseguide, which is configured to not be overflown during normal use of the microfluidic structure or during initial filling of the microfluidic structure with a first fluid.

In one example, the capillary pressure barrier comprises or consists of a rim or ridge of material protruding from an internal surface of the cell culture chamber and/or microfluidic channel; or a groove in an internal surface of the cell culture chamber and/or microfluidic channel. The sidewall of the rim or ridge may have an angle α with the top of the rim or ridge that is preferably as large as possible. In order to provide a good barrier, the angle α should be larger than 70°, preferably larger than 90°. The same counts for the angle α between the sidewall of the ridge and the internal surface of the culture chamber or microfluidic channel on which the capillary pressure barrier is located. Similar requirements are placed on a capillary pressure barrier formed as a groove.

An alternative form of capillary pressure barrier is a region of material of different wettability to an internal surface of the cell culture chamber or microfluidic channel, which acts as a spreading stop due to capillary force/surface tension. In one example, the internal surfaces of the cell culture chamber or microfluidic channel comprise a hydrophilic material and the capillary pressure barrier is a region of hydrophobic, or less hydrophilic material. In one example, the internal surfaces of the cell culture chamber or microfluidic channel comprise a hydrophobic material and the capillary pressure barrier is a region of hydrophilic, or less hydrophobic material.

Thus, in a particular embodiment of the present invention, the capillary pressure barrier is selected from a rim or ridge, a groove, a hole, or a hydrophobic line of material or combinations thereof. In other embodiments capillary pressure barriers can be created by a widening of a microfluidic channel or by pillars at selected intervals, the arrangement of which defines the first subvolume or area that is to be occupied by the droplet. In one example, the pillars extend the full height of the microfluidic channel.

As a result of the presence of a capillary pressure barrier, liquid, e.g. the droplet comprising cells or cell aggregates, is prevented from flowing beyond the capillary pressure barrier and enables the formation of stably confined volumes of liquid in the microfluidic structure, for example in one or more of the subvolumes inside the cell culture chamber and/or microfluidic channels.

The one or more capillary pressure barriers may bisect the fluid communication between the first and second reservoirs via the cell culture chamber. By providing a microfluidic structure having one or more capillary pressure barriers, a multiple network of microfluidic channels can be provided in order to pattern a cell culture medium, while allowing for fluid-fluid interaction between two or more channels, e.g. subvolumes. The one or more capillary pressure barriers may be designed such that the subvolumes may interact with each other to exchange medium contained in the subvolumes and/or components comprised in the medium.

In an embodiment of the present invention, the one or more capillary pressure barriers are arranged in the cell culture chamber in order to provide one or more subvolumes in the cell culture chamber. Such formation of one or more subvolumes even further increases the complexity of the network of microfluidic channels comprised in the microfluidic structure of the present invention. It is noted that the one or more subvolumes may be in fluid communication with the first and second reservoir of the at least one microfluidic structure of the present invention. However, alternatively, the one or more subvolumes may be in fluid communication with one or more further reservoirs comprised by the at least one microfluidic structure.

In order to avoid rupture, irregularities, disruptions or the like of cell culture medium or cultured cells (e.g. aggregates) in the subvolumes of the microfluidic structure comprising one or more further reservoirs, the one or more further reservoirs of the at least one microfluidic structure may be in fluid communication with another reservoir of the at least one microfluidic structure via a further communication channel (i.e. an additional communication channel in addition to the communication channel as defined above). Such further communication channel is preferably designed to have a pressure neutralizing effect on any pressure difference build-up in the one or more further reservoirs. Thus, preferably the fluidic resistance through the further communication channel between the one or more further reservoirs is lower than the fluidic resistance through the one or more subvolumes between the one or more further reservoirs. Said fluidic resistance is inversely related to the average cross-section of the further communication channel and related to the length of the further communication channel. Preferably, the fluidic resistance through the further communication channel is at least 5 times lower than the fluidic resistance through the one or more subvolumes, more preferably at least 10 times lower, even more preferably at least 50 times lower.

The difference in fluidic resistance can be estimated by applying the Navier-Stokes equation, the Hagen-Poiseuille law or a combination thereof. It is also possible to empirically determine the fluidic resistance by blocking the communication channel and measuring flow through the cell culture chamber at a predefined pressure, followed by blocking the cell culture chamber and measuring the flow through the communication channel at the same pressure. The difference in fluidic resistance through the cell culture chamber and through the communication channel should preferably be 5-fold, more preferably 10-fold, more preferably still 50-fold.

In one embodiment of the invention the microfluidic structure is configured such that the further communication channel is filled with a fluid of lower viscosity than the fluid in de cell culture chamber, e.g. a gaseous fluid. For example, the further communication channel can be positioned higher than the cell culture chamber in the normal operating orientation, so that gravity will ensure that the gaseous fluid fills the further communication channel while liquid fills the cell culture chamber.

The cell culture chamber comprised in the microfluidic structure of the present invention may be any kind of chamber suitable for culturing cells and/or tissue or the like. In an embodiment of the present invention, the microfluidic cell culture system comprises one or more cells and/or tissues. The cell culture chamber, but also the first and second reservoirs, may further be provided with a reversible solidifying medium comprising a solidifying agent and an aqueous medium.

The reversible solidifying medium is added in liquid form to the reservoirs and allowed to enter the cell culture chamber, after which the reversible solidifying medium is allowed to solidify. It is recognized that the solidified solidifying medium protects the cell culture in the cell culture chamber against external influences, e.g. against pressure fluctuations during transport.

In order to provide a solidifying medium wherein the medium once solidified is able to return to a liquid state, the percentage of mass of the solidifying agent compared to the total mass of the reversibly solidifying medium is about 1% to about 10%. Preferably, the percentage of mass of the solidifying agent compared to the total mass of the reversibly solidifying medium is about 2% to about 8%. More preferably, the percentage of mass of the solidifying agent compared to the total mass of the reversibly solidifying medium is about 3% to about 6%.

Preferred solidifying agents may be selected from the group consisting of gelatine, agar, xanthan gum, alginate and other non-Newtonian fluids. A preferred solidifying agent comprises gelatine.

The reversible solidifying medium is used to protect the contents of the microfluidic cell culture system against pressure fluctuations while under transport. After transportation, the reversible solidifying medium is removed from the system by first melting and subsequently aspirating it. Care should be taken not to heat the system too much, as temperatures higher than approximately 40° C. might damage the cell culture inside the cell culture chamber.

When selecting a suitable solidifying agent, the melting temperature of the resulting solution in water should preferably not be higher than 40° C. Nor should it be too low as this might lead too melting of the reversible solidifying medium during transport.

As such the solidifying agent can be selected for optimal compatibility with different shipping temperatures. When configuring for shipment at room temperature, i.e. typically approximately between 18° C. to 24° C., the preferred melting temperature of the reversible solidifying medium is between 32° C. to 40° C., more preferably between 35° C. to 38° C. When configuring for cold shipment at approximately 4° C., e.g. on wet ice or in refrigerated transport, the typically preferred melting temperature of the reversible solidifying medium is between 8° C. to 40° C., more preferably between 12° C. to 24° C.

For the selection of the optimal solidifying agent it is well-known that such selection depends on several parameters and is advised in the field with some caution as in particular situations some of the solidifying agents may result in a solidifying medium showing significant hysteresis in the melting temperature, i.e. the transitioning from a liquid phase to a solidified phase at a different temperature than the temperature at which the solidified medium transitions to a liquid phase. Also, in other situations some of the solidifying agents may result in a solidifying medium having a broader melting trajectory. For each of these configurations solidification should preferably occur above 0° C., and melting should preferably occur above 7° C., more preferably above 24° C., still more preferably above 32° C., but preferably below 40° C.

It should be noted that the preferred solidifying agent can be selected for optimal ease of use, compatibility with the temperature preference of the content of the cell culture system and robustness, by adjusting the melting and solidifying temperature to the typical temperatures expected during shipment, handling and in culture.

The communication channel comprised in the microfluidic cell culture system of the present invention may be any kind of communication channel arranged between the reservoirs of the microfluidic structure suitable for providing fluid communication between the reservoirs of the microfluidic structure. In an embodiment the communication channel comprises a passage provided in the at least one microfluidic structure. In a further embodiment the communication channel comprises a recess provided in a surface of the at least one microfluidic structure facing the inner side of the detachable seal. Both the passage and the recess are configured for connecting in fluid communication the reservoirs of the at least one microfluidic structure. Alternatively, the detachable seal is configured to airtight seal the at least one microfluidic structure and such that the inner side of the seal is configured to be located at some distance from the at least one microfluidic structure. In such way the inner side is located at some distance from the reservoirs (i.e. the opening of the reservoir facing the inner side of the detachable seal) of the at least one microfluidic structure providing a gap between the microfluidic structure and therewith providing a communication channel between the reservoirs of the microfluidic structure.

In an embodiment of the microfluidic cell culture system of the present invention, the microfluidic cell culture system comprises a plurality of microfluidic structures, thus forming a microfluidic microtiter plate comprising a plurality of microfluidic structures. Each of the microfluidic structure comprised in the microfluidic cell culture system comprising a plurality of microfluidic structures comprises at least two reservoirs in fluid communication with each other via a cell culture chamber comprised in the microfluidic structure. In order to prevent rupture, irregularities, disruptions or the like of cell culture medium, cultured cells (e.g. aggregates) in the cell culture chamber during transport of the microfluidic cell culture system comprising a plurality of microfluidic structures, the at least two reservoirs are also in fluid communication with each other via a communication channel as defined above. In addition, one or more of the reservoirs of one microfluidic structure may be in fluid communication with one or more reservoirs of one or more of the other microfluidic structures. Thus, forming an interconnected communication network of fluidly communicating reservoirs of different microfluidic structures. In addition, such interconnected communication network of fluidly communicating reservoirs of different microfluidic structures may be divided into a network comprising several subnetworks of a select number of different microfluidic structures in fluid communication with each other. In order to provide such a combination of subnetworks, a grid structure may be provided between the detachable seal and the plurality of microfluidic structures. The grid structure defining a plurality of grid sections wherein each grid section encloses one or more complete microfluidic structures. It was found that by providing a grid structure, the robustness of the microfluidic cell culture system during transport is further improved.

In a preferred embodiment of the microfluidic cell culture system of the present invention, the microfluidic cell culture system is configured such that the plurality of microfluidic structures are sealed by one detachable seal. Instead of using a plurality of different detachable seals, a single detachable seal is preferred, e.g. a cover plate used for covering the wells of a microfluidic microtiter plate.

In order to comply with the dimensions of the standard microtiter plate format, the at least one microfluidic structure may be dimensioned such that it corresponds to one or more wells of a microtiter plate or a cluster of wells of a microtiter plate. Preferably the at least one microfluidic structure may be dimensioned to correspond with one or more wells of a microtiter plate having 96, 384, or 1536 wells.

The detachable seal may comprise, at least partially, a semipermeable barrier configured to allow exchange from the microfluidic cell culture system to its external environment and/or vice versa. The semipermeable barrier may be configured to be impermeable for one or more predefined substances or quantities such as a gaseous medium, humidity, heat, moisture, a particle, a microbe, electricity, radiation and/or a virus. In addition, or alternatively, the detachable seal may comprise a vent, preferably a one-way vent, for providing fluid communication from the reservoirs of the at least one microfluidic structure to the external environment of the microfluidic cell culture system and/or vice versa.

In addition to sealing the microfluidic cell culture system using a detachable seal, the microfluidic cell culture system may be further sealed by a sealing packing. Such sealing packing may be configured to insulate and/or protect the microfluidic cell culture system from external influences, such as temperature fluctuation, mechanical forces, electricity, penetration, damage, contamination and/or moisture.

In addition, the microfluidic cell culture system may be packed in compliance with relevant laws and/or guidelines for transport of hazardous materials. In an embodiment of the present invention the microfluidic cell culture system is packed in compliance with the UN3373 standard. Preferably the microfluidic cell culture system is packed in a leak proof bag together with an absorbent material preferably further contained in a leak proof and shock absorbent further receptacle.

In a further aspect of the present invention the invention relates to a method for transporting microfluidic cell culture systems, the method comprising the steps of:
providing one or more microfluidic structures, wherein the one or more microfluidic structures optionally comprise an extracellular matrix and/or cells or cell aggregates, and wherein the microfluidic structures optionally comprise a solidified solidifying medium;
sealing the one or more microfluidic structures to form the microfluidic cell culture system according to the invention; and
transporting the sealed microfluidic cell culture system.

According to the method of the present invention, in case a solidifying medium is present, the solidifying medium may replace all or part of any cell culture medium present in the microfluidic structure.

The method of the present invention may further comprises the step of:
after transporting the microfluidic cell culture system, unsealing the one or more microfluidic structures;
optionally, allowing the solidified solidifying medium inside the one or more microfluidic structures to liquefy;
optionally, adding fresh cell culture medium to the reservoirs.

The method is based on the insight that although the microfluidic structure is sealed, i.e. with a detachable seal, a communication channel provided between the reservoirs will balance any pressure fluctuation that occurs during transport in the microfluidic structure. Advantageously, a reversible solidifying medium is added to the microfluidic cell culture system. It is added in liquid form and then allowed to solidify. The solidified solidifying medium provides additional protection against pressure fluctuations during transport.

Normally, the reversible solidifying medium is added to a microfluidic cell culture system that comprises a cell culture, e.g. a tube comprising CaCo-2 tubes, and is perfused with a cell culture medium. To prepare the microfluidic cell culture system for transport, the cell medium is first removed from the reservoirs, after which the liquefied solidifying medium is added to the reservoirs and allowed to solidify. After transport, the solidifying medium is carefully re-liquefied and removed from the reservoirs. After this step, cell culture medium is added to the reservoir in order to start again with perfusion of the cell culture inside the cell culture chamber.

Alternatively, removal of the cell medium can be omitted, or performed only partially, after which the reversible solidifying medium is added into the device in addition to the remaining cell culture medium. This can result in the solidifying agent mixing with the cell culture medium, still leading to solidification of all of the medium. This can also result in a part of the volume of medium solidifying while another part of the volume remains liquid. In a preferred embodiment, cell culture medium is removed from the reservoirs, but not from the cell culture chamber. The reversible solidifying medium is added to the reservoirs and allowed to solidify, resulting in solidified medium in the reservoirs shielding the unsolidified cell culture medium in the cell culture chamber from external influences, potentially resisting flow and/or displacement. It shall be noted that such configuration can be easily adjusted to achieve any configuration of subvolumes being solidified and liquid by removal and addition of cell culture medium and reversible solidifying medium in any order.

Extracellular matrices are well known to the person skilled in the art and can be any suitable substance or combination of substances, e.g. a hydrogel, e.g. comprising collagen I.

In an even further aspect of the present invention the invention relates to a kit-of-parts comprising one or more microfluidic structures and a seal. The one or more microfluidic structures and the seal are configured such that in assembled form the microfluidic cell culture system of the present invention is formed.

### EXAMPLES

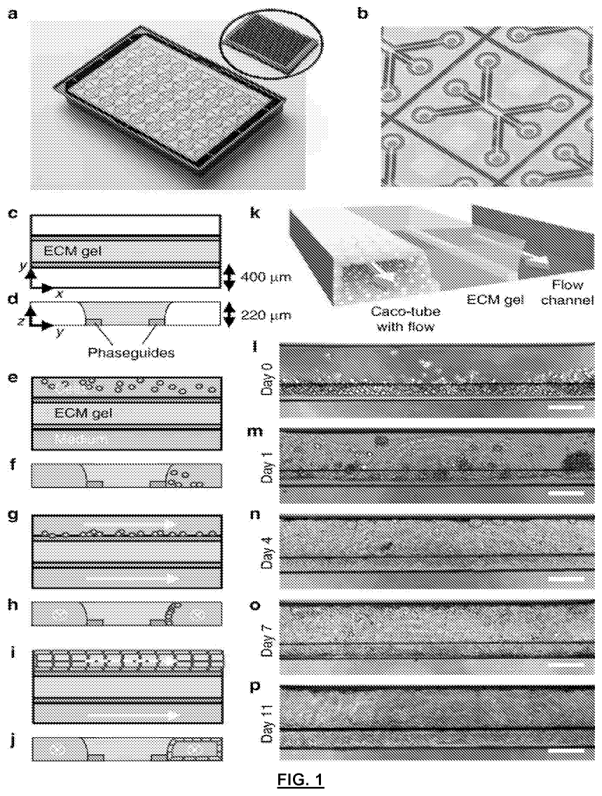

FIG. 1. Modelling intestinal tubules using the OrganoPlate platform (Trietsch et al. NComms, 2017)

The OrganoPlate® platform encompasses 40 microfluidic cell culture structures embedded in a standard 384-well microtiter plate format (FIGS. 1a and b, Trietsch et al. NComms, 2017) Trietsch et al. Lab Chip, 2013, Wevers et al. Sci. Rep., 2016. Each microfluidic channel structure is comprised of three lanes that are connected to corresponding wells of a microtiter plate that function as inlets and outlets to access the microfluidic culture. The lanes join in the centre of the structure where two capillary pressure barriers are present called phaseguides (Vulto et al. Lab Chip, 2011). FIG. 1c-j, Trietsch et al. NComms, 2017 shows a schematic representation of vertical and horizontal cross-sections of the centre of a microfluidic structure and the method of growing a tubular structure. First, an ECM gel is introduced in the central lane (FIG. 1c, d, Trietsch et al. NComms, 2017). The phaseguides are used to selectively pattern the ECM gel in the central lane by meniscus pinning. The meniscus stretches beyond the phaseguide, leading to a curved shape. After ECM gelation, epithelial cells are seeded in one lateral lane, allowing them to sediment directly against the ECM gel by placing the titre plate in a vertical position, i.e., standing on one side (FIG. 1e-h, Trietsch et al. NComms, 2017). Upon attachment of the cells, the plate is horizontally placed on an interval rocker that induces flow by reciprocal levelling between reservoirs. Upon application of flow, cells proliferate and start lining all surfaces of the perfusion channel, forming a confluent tubular structure (FIG. 1i, j, Trietsch et al. NComms, 2017). The tubules have a lumen that is connected to the in- and outlet of the respective lanes, making them accessible for perfusion with medium and for apical compound exposure. The basal side of the epithelium is facing the ECM gel and can be accessed by the second perfusion lane on the opposite side of the ECM gel lane. FIG. 1k Trietsch et al. NComms, 2017 depicts an artist impression of the 3D configuration of the tube, showing that the tube is grown directly against the ECM, without the presence of artificial membranes. For modelling of the intestinal barrier, the human intestinal colorectal adenocarcinoma cell line (Caco-2) was used. FIG. 1l-p, Trietsch et al. NComms, 2017 shows phase-contrast pictures of tube formation taken from the observation window well at day 0, 1, 4, 7, and 11, respectively. On day 0, cells are seeded against the ECM and start colonizing the glass walls to form a confluent tube (FIG. 1n-p, Trietsch et al. NComms, 2017). Perfusion was crucial for tube formation.

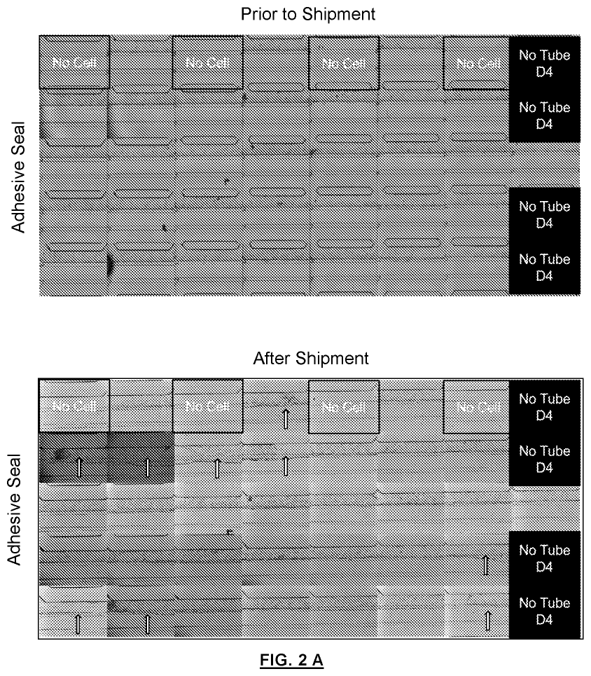

FIG. 2. Shipment of CaCo-2 cultures in an OrganoPlate® from Mimetas Leiden to Mimetas US located in Maryland, United States.

Plates were transported the industry standard way with microtiter plate seals. Brightfield images were used to capture before and after shipment state of the cultures. The CaCo-2 cultures were grown in the OrganoPlate® as previously described (adapted from Trietsch et al. NComms, 2017). The tube cultures were captured with brightfield imaging using an automated imaging system (Molecular Devices, ImageXpress Pico) and then prepared for shipment. This required preparing a 2.5% gelatine-medium solution by dissolving 2.5 g of gelatine powder (Sigma G9391) per 100 mL of CaCo-2 medium, and filtering with 0.22 μ filter once dissolved. All medium in the inlets/outlets of the OrganoPlate® was aspirated, 40 μL of the warmed gelatine-medium was added to each inlet/outlet. An adhesive clear seal (VWR catalogue 391-1251) was placed on top and pressed to complete sealing of all individual wells across the plate. The plate was then placed into a box for shipment. Upon arrival, brightfield images were taken at Mimetas US with an automated imaging system (BioTek, Cytation 1 Cell Imaging Reader) and compared with those taken prior to packaging at Mimetas Leiden (FIG. 2A). A closer view at single chip images show the difference in tube morphology prior to and after shipment (FIG. 2 B). With this packaging method, there were a number of CaCo-2 tubes that were damaged, visible by tube collapse or tube expansion into the ECM lane as indicated by the arrows (FIG. 2A-B).

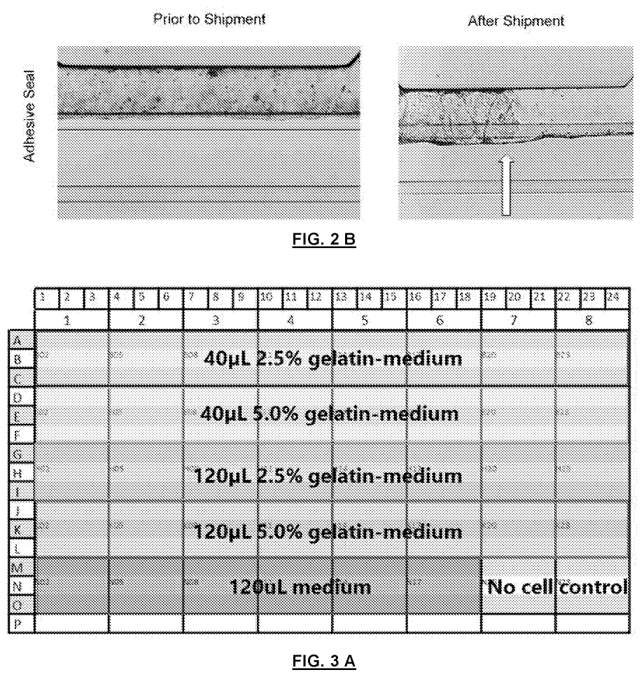

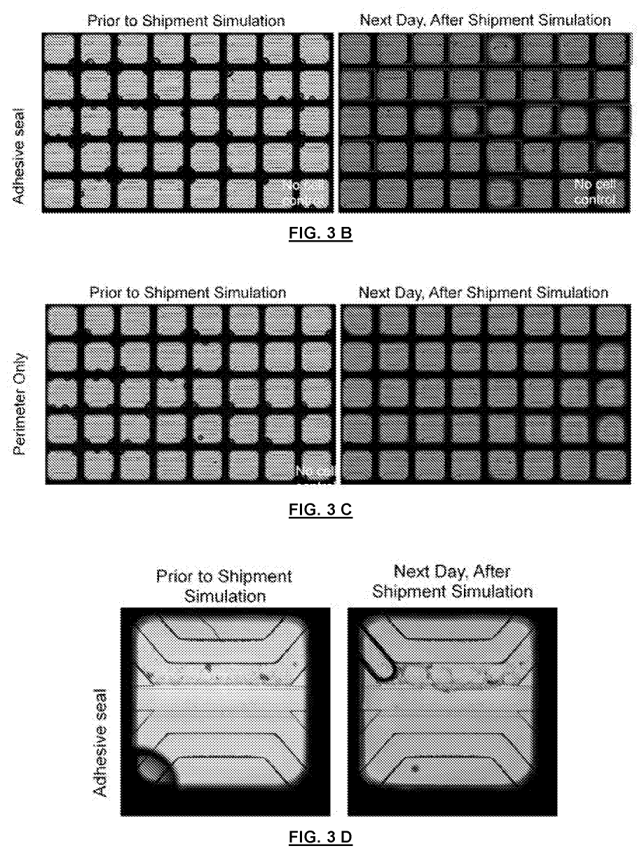

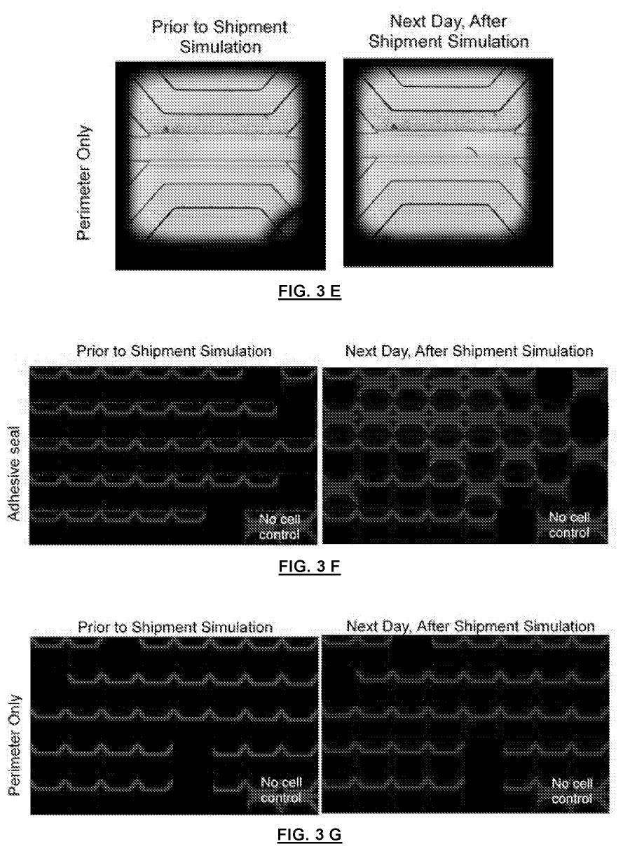

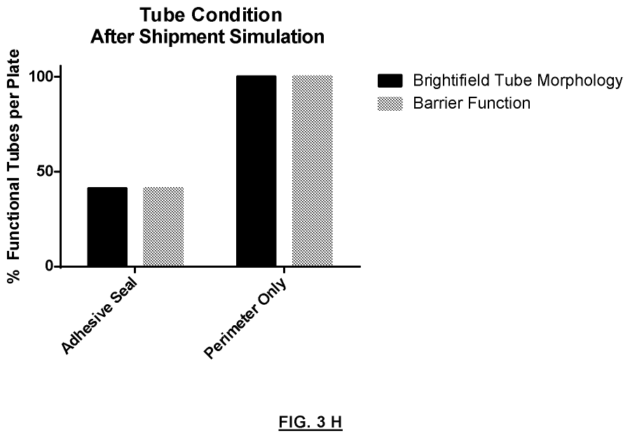

FIG. 3. Creation of a secondary route of fluid communication for the microfluidic chips.

The secondary rout of communication allows uniform equilibration of potential pressure differences during transport. To simulate shipment via air cargo transport, a low-pressure chamber was set up using a vacuum pump and airtight Tupperware. The tube cultures were generated as previously described and prior to shipment simulation their morphology was assessed visually with brightfield imaging and their functionally was assessed using the barrier integrity assay (Trietsch et al. NComms, 2017, WO2017/007325A1). To do this, 4.4 kDa TRITC-Dextran (Sigma-Aldrich T1037) diluted into medium at 0.5 mg/mL was added to all top channels and the observation window was imaged with TRITC microscope filter after t=15 minutes on an automated imaging system. To process for packaging, all inlets/outlets were aspirated, and different medium solutions were added to each OrganoPlate® inlets/outlets as follows (FIG. 3A): Row 1: 40 μL of 2.5% gelatine-medium prepared as described above, Row 2: 40 μL 5% gelatine-medium prepared as above with 5 g/100 mL of medium, Row 3: 120 μL 2.5% gelatine-medium prepared as above, Row 4: 120 μL 5% gelatine-medium prepared as above with 5 g/100 mL medium, Row 5: 120 μL medium. The observation windows of the plates were brightfield imaged using transmitted light on the ImageXpress Pico automated microscope, prior to shipment. One OrganoPlate® was sealed with an adhesive clear seal as done previously, while the other was sealed only at the perimeter of the lid, without separating individual wells. Both plates were then placed into the vacuum chamber. The shipping simulation was run by decreasing the pressure in the chamber to 800 mBar for 18 minutes to induce pressure changes, and then returned to atmospheric pressure and room temperature overnight to simulate the continued time in ground transit. The following morning, brightfield images were captured and another barrier integrity assay was run with the ImageXpress Pico as previously described. Both the brightfield (FIGS. 3 B-E) and t=15 minute TRITC fluorescent images (FIG. 3 F-G) acquired before and after shipment were compared. The images of the plate with individually sealed wells (FIG. 3 B, D, F) from after simulated shipment show both damaged Caco-2 tubes and bubbles in the chips (FIG. 3 B, D) in addition to decrease barrier function observed by permeation of TRITC-dextran to the ECM channel (FIG. 3 F). The images of the plate with perimeter seal showed no visible displacement or damage to the Caco-2 tube structure (FIG. 3 C, E), and little change to the barrier function (FIG. 3 G). These observations are quantified as a percentage of functional tubes relative to the number of functional tubes on that plate prior to shipment simulation (FIG. 3 H), where there is a clear decrease in both morphologically intact and functional barrier tubes with the adhesive sealed plate. This study confirms that the disruption of the CaCo-2 culture in the microfluidic plate under a change in pressure is due to the seal, where fluid communication is only allowed through the microfluidic channels themselves. With only a perimeter seal, fluid communication is possible through the top of the well and any pressure difference can equilibrate via this route to maintain tissue structure in the chip.

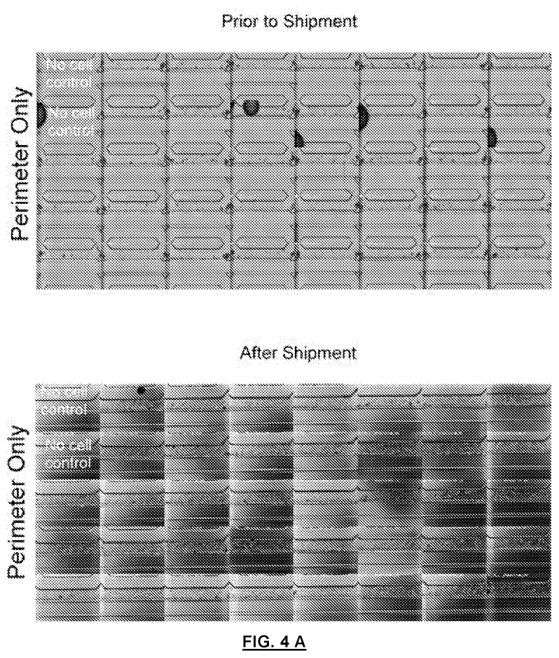

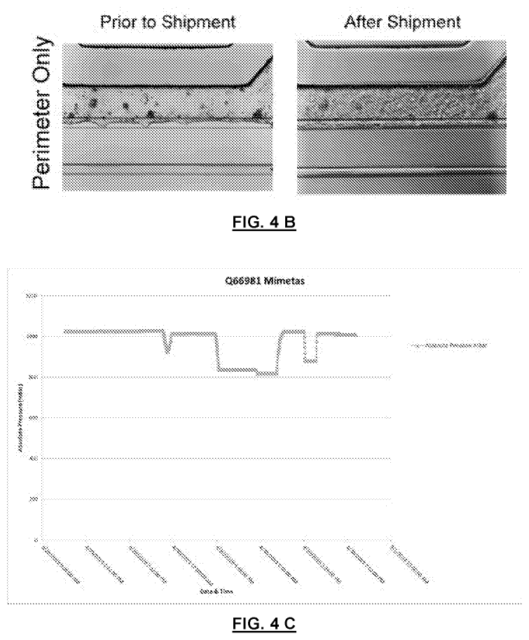

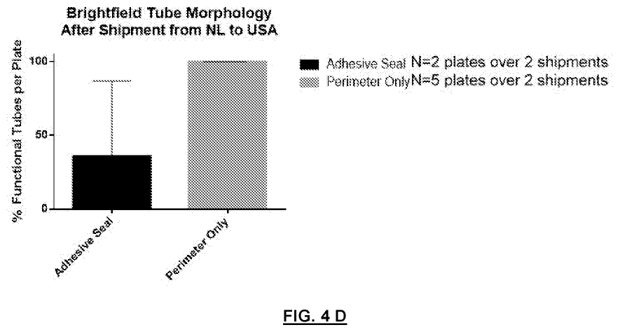

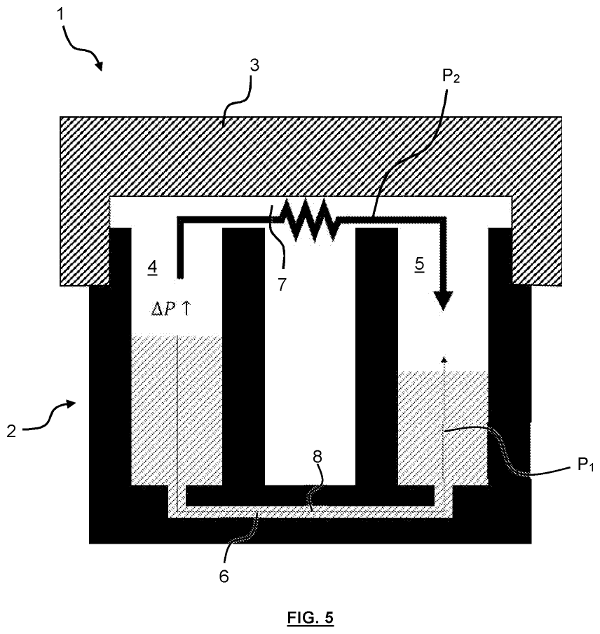

FIG. 4. Creation of a secondary route of fluid communication for the microfluidic chip.

Results of the proposed method with a real shipment. To confirm the feasibility of real life shipping, without individually sealing all reservoirs of a cell culture device, a shipment of CaCo-2 cultures in an OrganoPlate® was sent from Mimetas Leiden to Mimetas US. CaCo-2 cultures were grown as described, packaged by aspirating all inlet/outlet medium and replacing with 40 μL 2.5% or 4% gelatine-medium. (Columns 1-4 2.5% Columns 5-8 4%). A perimeter seal was applied to the device, which sealed the reservoirs of the device from outside influences, but allowed fluid communication between the wells. Medium compositions were chosen to maintain a gelled solution that would not spill out of the plate during normal transport forces. It should be noted that while the gelled solution is able to withstand inertial forces to prevent spillage, such gelled solution is typically not able to withstand the pressure differences induced by pressure changes associated with shipment of individually sealed reservoirs, as shown above by displacement or other distortion of the ECM and cells. Brightfield images were captured with the ImageXpress Pico automated microscope before at Mimetas Leiden, and after shipment to Mimetas US with the Cytation 1 automated microscope. The package was sent with a pressure data logger (MadgeTech, PRHTemp101A) to record absolute pressure. Comparing the before and after images, there was no damage, displacement, or trapped bubbles found in the plate upon receiving (FIG. 4A-B). The data logger did indicate several fluctuations in pressure and reached a minimum of 818 mBar during the transport (FIG. 4 C). Comparing different plates and shipments from The Netherlands to the US, the proposed method results in a higher percentage of functional tube tissues than the adhesive seal method following a real courier shipment (FIG. 4 D). This confirms that using gelled medium within the reservoirs of a microfluidic cell culture system, where multiple reservoirs are in fluid communication with each other forms a feasible method to ship microfluidic titerplates.

FIG. 5 shows a schematic view of the microfluidic cell culture system 1 comprising one microfluidic structure 2 sealed by a detachable seal 3. The microfluidic structure 2 comprises a first reservoir 4 and a second reservoir 5. Both reservoirs 4, 5 are in fluid communication via a cell culture chamber 6 as well as via a communication channel 7. The cell culture chamber 6 is filled with a cell culture medium 8. The volume of the cell culture medium 8 is chosen such that the cell culture chamber 6 is completely filled with the medium 8 as such. By providing a communication channel 7 between the first reservoir 4 and the second reservoir 5, a sudden increase in pressure (ΔP) in one of the reservoirs is easily balanced via the communication channel 7, instead of resulting in a sudden increase in pressure onto the cell culture medium 8 comprised in the cell culture chamber 6. The fluidic resistance of the different media used in the cell culture chamber 6 and the communication channel 7 is depicted in FIG. 5 by the thickness of the arrows shown in FIG. 5. The fluid communication lines between the first reservoir 4 and the second reservoir 5 is shown by a first arrow P₁ passing through the cell culture chamber 6 and a second arrow P₂ passing through the communication channel 7. The fluidic resistance of the medium 8 in the cell culture chamber 6 is significantly higher than the fluidic resistance of the medium in the communication channel 7 resulting in a major flow of medium from the first reservoir 4 to the second reservoir 5 via the communication channel 7 by an increased pressure in the first reservoir 4.

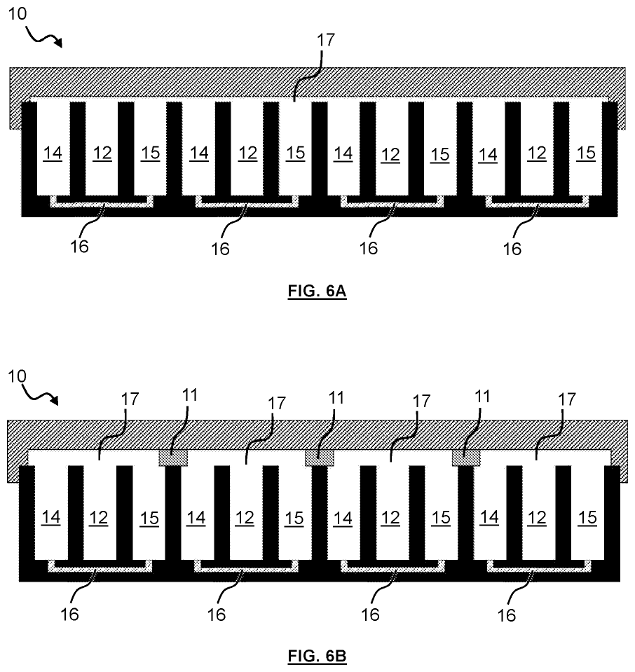

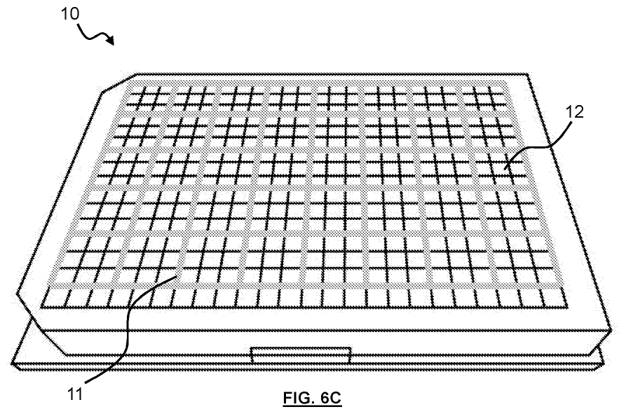

FIGS. 6A and 6B show a schematic view of the microfluidic cell culture system 10 comprising a plurality of microfluidic structures 12 sealed by a detachable seal 13. Each of the microfluidic structures 12 comprise a first reservoir 14 and a second reservoir 15 in fluid communication with each other via a cell culture chamber 16. The first and second reservoirs 14, 15 of the microfluidic structures 12 are further in fluid communication with each other via central communication channel 17 formed by a gap between the inner side of the seal 13 and the upper opening of each of the reservoirs 14, 15. Again any pressure increase in one of the reservoirs 14, 15 is easily balanced by providing a major flow of pressure trough the communication channel 17 instead of through one or more of the cell culture chambers 16 comprised in the microfluidic structures 12. In FIG. 6B the microfluidic cell culture system 10 is further provided with a grid structure 11, wherein the grid structure 11 encloses one or more complete microfluidic structures 12. In FIG. 6N the grid structure 11 encloses one single microfluidic structure 12.

FIG. 6C shows a perspective view of the microfluidic cell culture system 10 of which the schematic view is shown in FIG. 6B. in FIG. 6C, the grid structure 11 as well as the microfluidic structure 12 are visualised.

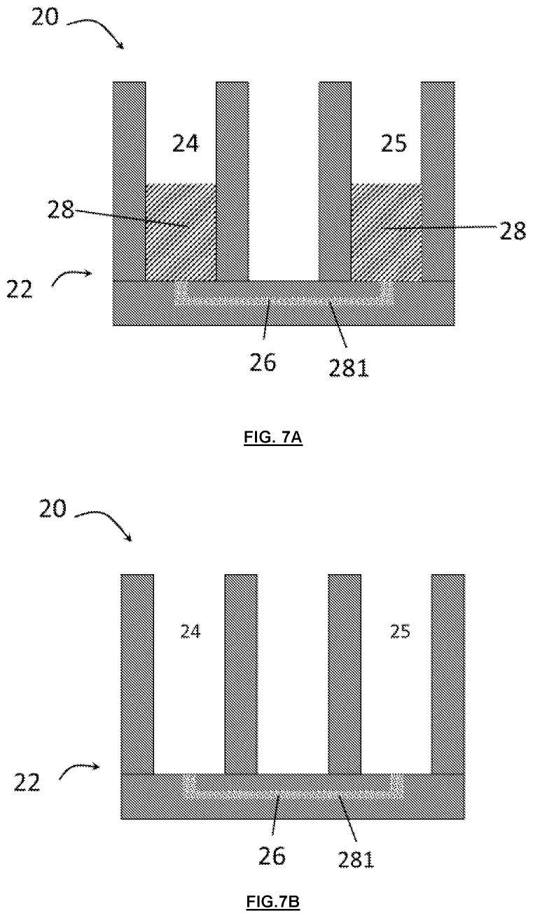

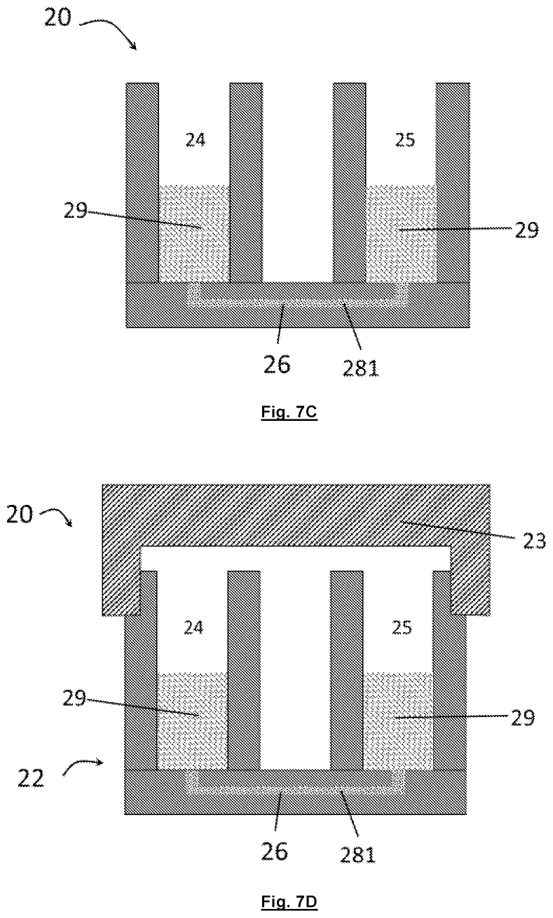

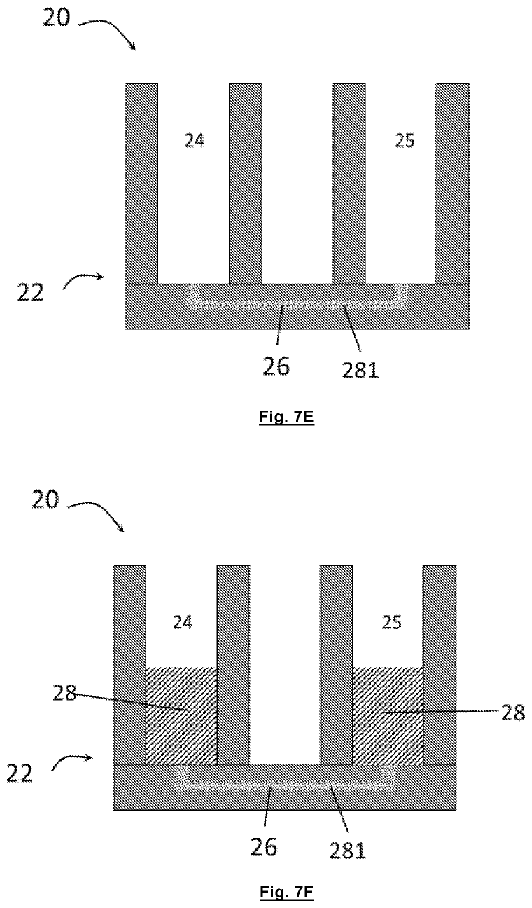

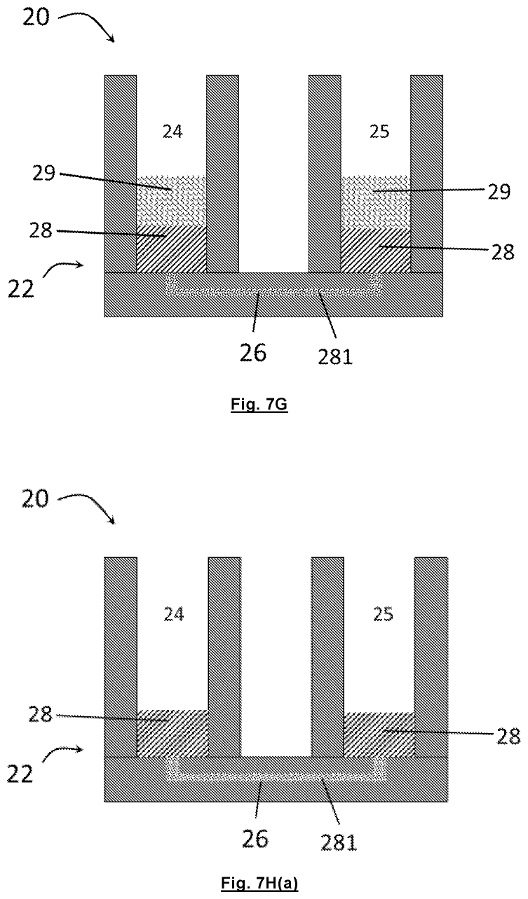

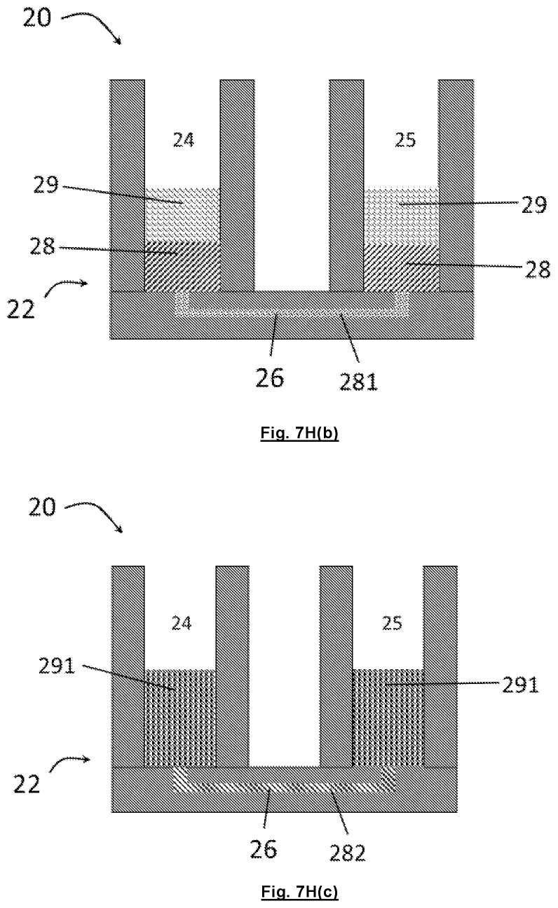

FIGS. 7A to 7F show schematic depictions of the steps in a method for transporting microfluidic cell culture systems. FIG. 7A shows a microfluidic cell culture system 20 comprising a cell culture chamber 26 in which cells are cultured and perfused against an extracellular matrix. The cells, e.g. CaCo-2 cells, are usually cultured in the form of a tubular structure or a tube (not shown). The combination of cells, extracellular matrix and cell culture medium is indicated in FIG. 7 with a dotted pattern fill and has the reference numeral 281. The microfluidic structure 22 also comprises reservoirs 24 and 25 that are filled with cell culture medium 28 for perfusing the tube inside the cell culture chamber 26. It is noted that the schematic drawing of FIG. 7A is highly simplified and does not show how exactly the medium 28 in the reservoirs 24, 25 enters the cell culture chamber 26 comprising the tube and the extracellular matrix. Advantageously, the microfluidic structure 22 has a layout as schematically shown in FIG. 1.

To prepare the microfluidic cell culture system 20 for transport, in a first step the medium 28 in the reservoirs 24, 25 is aspirated (FIG. 7B).

In the next step, shown in FIG. 7C, a warm liquefied reversible solidifying medium 29, e.g. a 2.5% gelatine solution, is added to the reservoirs 24, 25, thereby filling the cell culture chamber 26. The reversible solidifying medium 29 is allowed to solidify and indicated with the shingle fill pattern. For simplicity, the contents of the cell culture chamber 26 are shown with a dotted fill pattern, indicating the cells and the extracellular matrix, although the reversible solidifying medium 29 can also be present in the cell culture chamber 26.

By capping the microfluidic cell culture system 20 with a detachable seal 23 the microfluidic structure is closed from the surroundings and a communication channel 27 between the reservoirs 24 and 25 is created (FIG. 7D). The whole system can now be transported to its destination. Any pressure fluctuations that occur during transport, e.g. as a consequence of the microfluidic cell culture system being at an altitude of 10 km in an airplane cargo bay, will be relieved through communication channel 27 and absorbed by the solidified reversible solidifying medium 29.

After transport, the detachable seal 23 is carefully removed and the microfluidic cell culture system 20 is heated to re-liquefy the reversible solidifying medium 29. Heating should be done with care so as to not to damage the cell culture inside the cell culture chamber 26. When said medium 29 turned liquid again it is aspirated from the microfluidic cell culture system 20 (FIG. 7E).

Fresh cell culture medium 28 is added to the reservoirs 24, 25 after which the cell culture chamber 26 and its contents 281 can be perfused again (FIG. 7F). Note that the step depicted in FIG. 7F shows the starting situation depicted in FIG. 7A, on a different location.

FIGS. 7G and 7H(a)-(c) schematically depict the methods in which the reversible solidifying medium 29 is added to reservoirs 24, 25 in addition to and on top of the cell culture medium 28. In FIG. 7G the situation is depicted where the reversible solidifying medium 29 solidifies quickly and does not mix with cell culture medium 28. FIGS. 7H(a)-(c) show 3 subsequent steps. FIG. 7H(a) shows the reservoirs 24, 25 comprising cell culture 28 and the cell culture chamber 26 comprises a mixture of cells, e.g. in the form of a tube, extracellular matrix and cell culture medium, the mixture denoted 281. The volume of cell culture medium can be reduced to prevent spilling when adding reversible solidifying medium 29. Step ii (FIG. 7H(b)) shows how reversible solidifying medium 29 is added to reservoirs 24, 25, in liquid form, whereas step iii (FIG. 7H(c)) shows the situation where the reversible solidifying medium 29 and the cell culture medium 28 have mixed into mixed medium 291 and solidified. Note that the liquid reversible solidifying medium 29 also mixes with the cell culture medium 28 present in the cell culture chamber 26, leading to a situation where the tube of cells and extracellular matrix are surrounded and infiltrated by mixed medium 291. For clarity reasons, the whole of cells, extracellular matrix and solidified mixed medium 291 is denoted mixture 282. Note that FIG. 7H(c) is a highly simplified schematic. In reality, there will be no clear demarcation line between mediums 291 and mixture 282.

1. A microfluidic cell culture system comprising at least one microfluidic structure, wherein the at least one microfluidic structure comprises:
a cell culture chamber for holding a medium for culturing cells, the cell culture chamber comprising a microfluidic inlet opening and a microfluidic outlet opening;
a first reservoir in fluid communication with the cell culture chamber via the microfluidic inlet opening; and
a second reservoir in fluid communication with the cell culture chamber via the microfluidic outlet opening,
wherein the microfluidic cell culture system further comprises a detachable seal for sealing the at least one microfluidic structure,
and wherein the first and second reservoir of the at least one microfluidic structure are in fluid communication with each other via the cell culture chamber,
characterised in that
the microfluidic cell culture system is configured such that the first and second reservoir of the at least one microfluidic structure are in fluid communication with each other via a communication channel that does not comprise the cell culture chamber, and in that the communication channel is bidirectional.

2. The microfluidic cell culture system according to claim 1, wherein the fluidic resistance through the communication channel between the first and second reservoir is lower than the fluidic resistance through the cell culture chamber between the first and second reservoir, preferably wherein the fluidic resistance through the communication channel is at least 5 times lower than the fluidic resistance through the cell culture chamber, more preferably at least 10 times lower, even more preferably at least 50 times lower.

3. The microfluidic cell culture system according to claim 1, wherein the communication channel is configured to exchange a gaseous medium, such as nitrogen, oxygen or air, between the first reservoir and the second reservoir.

4. The microfluidic cell culture system according to claim 1, wherein the microfluidic structure comprises one or more capillary pressure barriers, such as phaseguides, in order to provide one or more subvolumes, and wherein the one or more capillary pressure barriers are arranged in the cell culture chamber, thereby providing one or more subvolumes.

5. (canceled)

6. (canceled)

7. The microfluidic cell culture system according to claim 4, wherein the one or more subvolumes are in fluid communication with the first and second reservoir of the at least one microfluidic structure or wherein the one or more subvolumes are in fluid communication with one or more further reservoirs comprised by the at least one microfluidic structure, wherein each of said one or more further reservoirs of the at least one microfluidic structure are in fluid communication with another reservoir of the at least one microfluidic structure via a further communication channel.

8. (canceled)

9. (canceled)

10. The microfluidic cell culture system according to claim 1, wherein the cell culture chamber comprises one or more cells and/or tissues.

11. The microfluidic cell culture system according to claim 1, wherein the first and second reservoirs and/or the cell culture chamber are provided with a reversible solidifying medium comprising a solidifying agent and an aqueous medium.

12. (canceled)

13. (canceled)

14. The microfluidic cell culture system according to claim 1, wherein the communication channel comprises a passage provided in the at least one microfluidic structure or a recess provided in a surface of the at least one microfluidic structure facing the inner side of the detachable seal wherein the passage and the recess are configured for connecting in fluid communication the first reservoir with the second reservoir of the at least one microfluidic structure.

15. The microfluidic cell culture system according to claim 1, wherein the detachable seal is configured to airtight seal the at least one microfluidic structure and wherein the detachable seal is configured to be located relative to the at least one microfluidic structure such that the inner side of the seal is located at some distance from the reservoirs of the at least one microfluidic structure.

16. The microfluidic cell culture system according to claim 1, wherein the microfluidic cell culture system comprises a plurality of microfluidic structures and wherein one or more of the reservoirs of one microfluidic structure is in fluid communication with one or more reservoirs of one or more of the other microfluidic structures.

17-20. (canceled)

21. The microfluidic cell culture system according to claim 1, wherein at least a part of the detachable seal comprises:
a semipermeable barrier configured to allow exchange from the microfluidic cell culture system to its external environment and/or vice versa, wherein the semipermeable barrier is configured to be impermeable for a one or more predefined substances or quantities such as a gaseous medium, humidity, heat, moisture, a particle, a microbe, electricity, radiation and/or a virus; and/or
a vent, preferably a one-way vent, for providing fluid communication from the reservoirs of the at least one microfluidic structure to the external environment of the microfluidic cell culture system and/or vice versa.

22-25. (canceled)

26. A method for transporting microfluidic cell culture systems, the method comprising the steps of:
providing one or more microfluidic structures, wherein the one or more microfluidic structures optionally comprise an extracellular matrix and/or cells or cell aggregates, and wherein the microfluidic structures optionally comprise a solidified solidifying medium
sealing the one or more microfluidic structures to form the microfluidic cell culture system according to claim 1; and
transporting the sealed microfluidic cell culture system

27. The method according to claim 26, wherein the solidifying medium replaces all or part of any cell culture medium present in the microfluidic structure.

28. The method according to claim 26, wherein the method further comprises the step of:
after transporting the microfluidic cell culture system, unsealing the one or more microfluidic structures;
optionally, allowing the solidified solidifying medium inside the one or more microfluidic structures to liquefy; and
optionally, adding fresh cell culture medium to the reservoirs.

29. A kit of parts comprising one or more microfluidic structures and a seal, wherein the one or more microfluidic structures and the seal are configured such that in assembled form the microfluidic cell culture system according to claim 1 is formed.
